(19) United States
(12) Patent Application Publication
(10) Pub. No.: US 2024/0016878 A1
(43) Pub. Date: Jan. 18, 2024

(54) COMPOSITION FOR PREVENTING, AMELIORATING OR TREATING ANDROGEN-DEPENDENT DISORDER COMPRISING PHYLLOSTACHYS PUBESCENS EXTRACT AS EFFECTIVE COMPONENT

(71) Applicant: KOREA INSTITUTE OF ORIENTAL MEDICINE, Daejeon (KR)

(72) Inventor: Kwang-Hoon SONG, Daejeon (KR)

(21) Appl. No.: 18/038,537

(22) PCT Filed: Oct. 27, 2020
(86) PCT No.: PCT/KR2020/014701

§ 371 Date: May 24, 2023

(30) Foreign Application Priority Data
Nov. 29, 2019 (KR) 10-2019-0156718

(51) Int. Cl. A61K 36/899; A61P 5/26; A61K 8/9794

(52) U.S. Cl. CPC A61K 36/899; A61P 5/26; A61K 8/9794; A61K 2236/331; A61K 2236/333

(57) ABSTRACT

A composition for preventing, ameliorating or treating an androgen-dependent disorder includes Phyllostachys pubescens extract as an effective component. It was found that, compared to the extracts of other types of bamboo, the Phyllostachys pubescens extract of the present invention can significantly reduce the expression amount of the SRD5A2 gene which encodes 5α-reductase while hardly showing any cytotoxicity. The composition of the present invention was found to have effects that can reduce the prostate weight, reduce the proliferation of epithelial cells in prostate tissues, and also can reduce the content of testosterone, dihydrotestosterone, PSA, and SRD5A2 in blood serum. Thus, the composition of the present invention can be advantageously used for an androgen-dependent disorder.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2020/014701, filed Oct. 27, 2020, which claims priority to the benefit of Korean Patent Application No. 10-2019-0156718 filed in the Korean Intellectual Property Office on Nov. 29, 2019, the entire contents of which are incorporated herein by reference.

### BACKGROUND

### 1. Technical Field

The present invention relates to a composition for preventing, ameliorating or treating an androgen-dependent disorder comprising Phyllostachys pubescens extract as effective component.

### 2. Background Art

Androgen is the generic term for any hormone which exhibits an influence on growth and development of a male reproductive system. Also called androgenic hormone, it refers to all substances exhibiting the activity of male hormones. As a hormone responsible for the production of secondary male sex characteristics, it is mainly secreted from the testes of a male while the adrenal cortex and female ovary also produce some androgens. Androgen is a steroid comprising 19 carbon atoms and it includes testosterone secreted by testes, dihydrotestosterone resulting from the reduction in cells, dehydroepiandrosterone or androsterone, which is excreted in urine after transformation from dihydrotestosterone, and adrenosterone secreted by the adrenal cortex.

Those hormones promote the development, maintenance, and activities of the reproductive organs or other sexual characteristics. In particular, they are involved in having high level of proteins in bone tissues, large weight and size of kidney, high activity of sweat and sebaceous glands, regeneration of red blood cells, or the like. On the skin, they cause thicker stratum corneum to yield the production of a large amount of sebum, thus becoming a reason for having teen acne during puberty. Body hair is also caused by the androgenic hormones. In the case of a male, beard growth tends to be stimulated by androgen while hair loss showing regression on the forehead or crown is also caused by the hormone.

Among the androgen-dependent disorders that are caused by the abnormally high level of androgen, benign prostatic hyperplasia (BPH) has been traditionally characterized as a condition showing weak urine stream resulting from urinary tract obstruction, i.e., blockage of a urinary tract at the base of the bladder as caused by an enlarged prostate. In terms of histology, BPH is defined as a condition in which hyperproliferation of stromal cells in the transition zones or epithelial tissues of the prostate gland is shown. In recent days, however, the symptomatic state of disorders is so complicated that it cannot be easily explained by the aforementioned definition or concept. Thus, BPH is currently defined as a condition showing lower urinary tract symptoms which include both the bladder storage symptom such as frequent urination like 8 or more times per day by men aged 50 years or older, frequent urination during night time, or urinary urgency with uncontrollable urge involving a sudden, strong need to urinate (i.e., feeling to have to pee) and symptoms showing a problem in excretion of the bladder such as delayed voiding (i.e., symptom with difficulty starting a urine stream), interrupted urination (i.e., symptom with intermittent urine stream), or a symptom requiring a strain or a push to urinate.

Major clinical characteristics of BPH include enlarged prostate and symptoms occurring in the lower urinary tract. Enlarged prostate is observed in the presence of androgen. It is known that anabolic steroids can increase the prostate size to reduce urine flow, consequently causing increased urinary frequency.

The prostate is an androgen-dependent organ in which testosterone and its extratesticular precursors are activated into more potent DHT (dihydrotestosterone). In general, the prostate plays a key role in producing DHT, and the systemic effect of the endocrine activity of a prostate is involved in the forming and secretion of DHT for its circulation. As people grow older, the production amount of DHT increases, causing overgrowth and enlargement of the prostate. The importance of DHT has been confirmed by the clinical studies in which α-reductase inhibitor is administered to a male patient with BPH. It is known that, in many cases, both the DHT level in prostate and prostate size can be significantly reduced by therapy using a 5α-reductase inhibitor. Finasteride is widely used for treating androgen-dependent disorders like male pattern baldness, benign prostatic hyperplasia (BPH), and prostate cancer. Finasteride is a competitive and specific 5α-reductase inhibitor, and, by blocking the conversion of testosterone to DHT in the prostate, hair follicles, and other androgen-sensitive tissues, it causes a decline in DHT concentration in blood serum and prostate. Although it has been proven that the conventionally used pharmaceuticals like finasteride and dutasteride are useful for a therapy effective for treating BPH, due to the side effects including erectile dysfunction, loss of libido, dizziness, and upper respiratory tract infection, use of the pharmaceuticals is now strictly regulated.

Meanwhile, Phyllostachys pubescens also referred to as Honam bamboo, Jooksoon bamboo, or Mo bamboo is characterized in that it is very thick and grows to 10 to 20 meters. It is mostly found in the southern area of the Korean peninsula and has dark brown spots on bamboo skin, which is very hard with little shine. As it has a problem like brittleness due to poor elasticity, hollow bamboo is often used by itself. It is known that, by containing silicic acid, terpene, and tannin, Phyllostachys pubescens has an anti-bacterial activity, and, with the generation of negative ions, it is useful for having uncontaminated blood, high appetite, calmness, and recovery from fatigue. It is also known that bamboo shoots are useful for recovery from hangovers, stress, and insomnia and also for helping urination and preventing constipation. It can be also useful for having an anti-cancer activity and preventing high blood pressure, arteriosclerosis, and chronic hepatitis, and, by containing a rich amount of fibers, it promotes intestinal peristalsis.

In Korean Patent Registration No. 0479021, a method for obtaining Phyllostachys pubescens extract is described, and, in Korean Patent Application Publication No. 2010-0066743, a composition for improving the state of hair and scalp comprising bamboo sap is described. However, so far there is no disclosure of a composition for preventing, ameliorating or treating an androgen-dependent disorder comprising Phyllostachys pubescens extract as an effective component as it is described in the present invention.

### SUMMARY

The present invention is devised under the circumstances that are described above, and the present invention provides a composition for preventing, ameliorating, or treating an androgen-dependent disorder comprising Phyllostachys pubescens extract as an effective component. It was found that, compared to the extracts of other types of bamboo, the Phyllostachys pubescens extract of the present invention can significantly reduce the expression amount of the SRD5A2 gene which encodes 5α-reductase while hardly showing any cytotoxicity. It was also found that, when an animal model of enlarged prostate is orally administered with the Phyllostachys pubescens extract of the present invention, the prostate weight is reduced, the proliferation of epithelial cells in prostate tissues is reduced, and also the content of testosterone, dihydrotestosterone, PSA, and SRD5A2 in blood serum is reduced, and the present invention is completed accordingly.

To achieve the purpose described above, the present invention provides a functional health food composition for preventing or ameliorating an androgen-dependent disorder comprising Phyllostachys pubescens extract as an effective component.

The present invention further provides a pharmaceutical composition for preventing or treating an androgen-dependent disorder comprising Phyllostachys pubescens extract as an effective component.

The present invention still further provides a cosmetic composition for preventing or ameliorating androgen-dependent alopecia comprising Phyllostachys pubescens extract as an effective component.

The present invention is devised under the circumstances that are described above, and the present invention relates to a composition for preventing, ameliorating, or treating an androgen-dependent disorder comprising Phyllostachys pubescens extract as an effective component. It was found that, compared to the extracts of other types of bamboo, the Phyllostachys pubescens extract of the present invention is effective in that it can significantly reduce the expression amount of SRD5A2 gene which encodes 5α-reductase while hardly showing any cytotoxicity. The Phyllostachys pubescens extract of the present invention is also effective in that when an animal model of enlarged prostate is orally administered with the extract, the prostate weight is reduced, proliferation of epithelial cells in prostate tissues is reduced, and also the content of testosterone, dihydrotestosterone, PSA, and SRD5A2 in blood serum is reduced.

### BRIEF DESCRIPTION OF THE DRAWINGS

FIG. 1 is a schematic drawing of the luciferase assay system.

FIG. 2 shows the result of the luciferase assay for analyzing a change in SRD5A2 expression in the hot water extract of Lophatheri herba, ethanol extract of Lophatheri herba, hot water extract of Bambusae caulis, hot water extract of Sasa borealis, and Phyllostachys pubescens (Phyllostachys pubescens Mazel) extract of the present invention. In FIG. 2, C represents a negative control that has been treated with a vehicle, 1 to 3 represent a positive control that been treated with (1) dutasteride (25 μM), (2) finasteride (50 μM), or (3) saw palmetto fruit extract (SPE, 100 μg/ml), all 5α-reductase inhibitor, 4 represents a group treated with 80% ethanol extract of 30 μg/ml Phyllostachys pubescens (Phyllostachys pubescens Mazel, Phyllostachys edulis, Phyllostachys pubescens), 5 represents a group treated with water extract of 30 μg/ml Lophatheri herba (Lophatherum gracile Brongn., Lophatherum gracile Bronghiart), 6 represents a group treated with 70% ethanol extract of 30 μg/ml Lophatheri herba, 7 represents a group treated with hot water extract of 30 μg/ml Bambusae caulis, and 8 represents a group treated with hot water extract of 30 μg/ml Sasa borealis.

FIG. 3 shows the result of the luciferase assay for analyzing a change in SRD5A2 expression in a group treated with 1 to 100 μg/ml Phyllostachys pubescens (Phyllostachys pubescens Mazel) of the present invention. In FIG. 3, C represents a negative control which has been treated with a vehicle, 1 to 3 represent a positive control which has been treated with (1) dutasteride (25 μl), (2) finasteride (50 μM), or (3) saw palmetto fruit extract (SPE, 100 μg/ml), all 5α-reductase inhibitor, 4 represents a group treated with 80% ethanol extract of 1 μg/ml Phyllostachys pubescens, 5 represents a group treated with 80% ethanol extract of 3 μg/ml Phyllostachys pubescens, 6 represents a group treated with 80% ethanol extract of 10 μg/ml Phyllostachys pubescens, 7 represents a group treated with 80% ethanol extract of 30 μg/ml Phyllostachys pubescens, and 8 represents a group treated with 80% ethanol extract of 100 μg/ml Phyllostachys pubescens.

FIG. 4 shows the result of determining the mRNA expression amount of SRD5A2 after treating BPH-1 cells with 3 to 30 μg/ml Phyllostachys pubescens extract for 24 hours, in which the determination was made by real-time PCR. In FIG. 4, C represents a negative control which has been treated with a vehicle, 1 to 3 represent a positive control which has been treated with (1) dutasteride (25 μM), (2) finasteride (50 μM), or (3) saw palmetto fruit extract (SPE, 100 μg/ml), all 5α-reductase inhibitor, 4 represents a group treated with 80% ethanol extract of 3 μg/ml Phyllostachys pubescens, represents a group treated with 80% ethanol extract of 10 μg/ml Phyllostachys pubescens, and 6 represents a group treated with 80% ethanol extract of 30 μg/ml Phyllostachys pubescens.

FIG. 5 shows the result of the MTS assay for determining the cytotoxicity of the Phyllostachys pubescens extract of the present invention. In FIG. 5, C represents a negative control which has been treated with a vehicle, 1 represents a control which has been treated with 10% DMSO, 2 to 4 represent a positive control which has been treated with (2) dutasteride (25 μM), (3) finasteride (50 μM), or (4) saw palmetto fruit extract (SPE, 100 μg/ml), all 5α-reductase inhibitor, 5 represents a group treated with 80% ethanol extract of 1 μg/ml Phyllostachys pubescens, 6 represents a group treated with 80% ethanol extract of 3 μg/ml Phyllostachys pubescens, 7 represents a group treated with 80% ethanol extract of 10 μg/ml Phyllostachys pubescens, 8 represents a group treated with 80% ethanol extract of 30 μg/ml Phyllostachys pubescens, 9 represents a group treated with 80% ethanol extract of 100 μg/ml Phyllostachys pubescens, and 10 represents a group treated with 80% ethanol extract of 300 μg/ml Phyllostachys pubescens.

FIG. 6 shows the result of determining the content of testosterone, dihydrotestosterone, PSA (prostate specific antigen), and SRD5A2 in blood serum after administering the Phyllostachys pubescens extract of the present invention. ### indicates that, compared to the control (CON), the BPH group has a statistically significant increase in the content of testosterone, DHT, PSA, and SRD5A2 (p<0.001). *, **, and *** indicate that compared to the BPH group, the group administered with the Phyllostachys pubescens extract of the present invention (PPE) has a statistically significant decrease in the content of testosterone, DHT, PSA, and SRD5A2 (*; p<0.05, **; p<0.01, and ***; p<0.001).

FIG. 7 shows the result of determining the weight of the prostate after administering the Phyllostachys pubescens extract of the present invention. ### indicates that, compared to the control, the BPH group which has been induced to have an enlarged prostate has a statistically significant increase in the weight of the prostate (p<0.001). * and *** indicate that, compared to the group induced to have enlarged prostate (BPH), the group administered with the Phyllostachys pubescens extract of the present invention (PPE) has a statistically significant decrease in the weight of prostate (*; p<0.05 and ***; p<0.001).

FIG. 8 shows the result of determining the thickness of prostate epithelial tissues after administering the Phyllostachys pubescens extract of the present invention. ### indicates that, compared to the control, the BPH group which has been induced to have an enlarged prostate has a statistically significant increase in the thickness of prostate epithelial tissues (p<0.001). *** indicates that, compared to the group induced to have an enlarged prostate (BPH), the group administered with the Phyllostachys pubescens extract of the present invention (PPE) has a statistically significant decrease in the thickness of prostate epithelial tissues (p<0.001).

FIG. 9 shows the result of determining a change in the TNFα content in prostate tissues after administering the Phyllostachys pubescens extract of the present invention. ### indicates that, compared to the control, the BPH group which has been induced to have an enlarged prostate has a statistically significant increase in TNFα content in prostate tissues (p<0.001). ** and *** indicate that, compared to the group induced to have enlarged prostate (BPH), the group administered with the Phyllostachys pubescens extract of the present invention (PPE) has a statistically significant decrease in the TNFα content in prostate tissues (**; p<0.01 and ***; p<0.001).

### DETAILED DESCRIPTION

The present invention relates to a functional health food composition for preventing or ameliorating an androgen-dependent disorder comprising Phyllostachys pubescens extract as an effective component.

The Phyllostachys pubescens extract can be produced by a method including the following steps:
(1) carrying out extraction by adding an extraction solvent to Phyllostachys pubescens;
(2) filtering the extract of the step (1); and
(3) concentrating under reduced pressure the filtered extract of the step (2) followed by drying to produce an extract,
but the method is not limited thereto.

The extraction solvent of the above step (1) is preferably selected from water, C₁-C₄ lower alcohol, and a mixture thereof. It is more preferably ethanol, and even more preferably 80% (v/v) ethanol, but not limited thereto.

With regard to the production method, any kind of common methods that are generally known as extraction methods in the pertinent art, e.g., filtration, hot water extraction, impregnation extraction, extraction by reflux condensation, and ultrasonic extraction, can be used. It is preferable that the extraction is carried out by adding an extraction solvent in an amount of 1 to 20 times the dry weight of Phyllostachys pubescens. More preferably, the extraction solvent is added in an amount of 5 to 15 times the dry weight of Phyllostachys pubescens. The extraction temperature is preferably between 20° C. and 90° C., but it is not limited thereto. Furthermore, the extraction time is preferably between 0.5 hours and 10 hours, more preferably between 3 hours and 7 hours, and most preferably 3 hours or 5 hours, but it is not limited thereto. It is preferable that the concentration of step (3) in the above method uses a vacuum rotary condenser or a vacuum rotary evaporator, but it is not limited thereto. Furthermore, the drying is preferably carried out by drying under reduced pressure, drying under a vacuum, drying under boiling, spray drying, freeze-drying, or microwave drying, but it is not limited thereto.

The Phyllostachys pubescens is preferably a leaf of Phyllostachys pubescens Mazel, but it is not limited thereto. The androgen-dependent disorder is preferably anyone selected from alopecia, acne, seborrheic dermatitis, prostatitis, benign prostatic hyperplasia, and dysuria caused by benign prostatic hyperplasia, but it is not limited thereto.

The functional health food composition for preventing or ameliorating an androgen-dependent disorder comprising Phyllostachys pubescens extract as an effective component can be prepared in any formulation selected from a drink, a pill, a tablet, a capsule, and a powdery or the preparation may be achieved by adding the extract as a component of food product, and it can be suitably produced by a general method.

Examples of the food to which the Phyllostachys pubescens extract of the present invention can be added include meat, sausage, bread, chocolate, candies, snacks, biscuits, pizza, ramen, other noodles, gums, dairy products including ice cream, various kinds of soup, beverage, tea, drink, alcohol beverage, and vitamin complex, and all functional health food products in the general sense are included therein.

The functional health food composition may further comprise various nutritional supplements, a vitamin, a mineral (i.e., electrolyte), a natural or synthetic flavor, a coloring agent, an enhancing agent (e.g., cheese and chocolate), pectinic acid and a salt thereof, alginic acid and a salt thereof, an organic acid, a protective colloidal thickening agent, a pH adjusting agent, a stabilizer, a preservative, glycerin, alcohol, and a carbonating agent used for carbonated drink. Other than those, fruit juice or fruit pulp for producing vegetable drinks may be additionally comprised. Those ingredients may be used either singly or in combination thereof.

The functional health food composition of the present invention may comprise various flavoring agents and natural carbohydrates as an additional component. Examples of natural carbohydrates include monosaccharides like glucose and fructose, disaccharides like maltose and sucrose, polysaccharides like dextrin and cyclodextrin, and sugar alcohols like xylitol, sorbitol, and erythritol. As a sweetening agent, natural sweetening agents like thaumatin and stevia extract and synthetic sweetening agents like saccharine and aspartame can be used.

The present invention further relates to a pharmaceutical composition for preventing or treating an androgen-dependent disorder comprising Phyllostachys pubescens extract as an effective component.

The androgen-dependent disorder is preferably any one selected from alopecia, acne, seborrheic dermatitis, prostatitis, benign prostatic hyperplasia, and dysuria caused by benign prostatic hyperplasia, but it is not limited thereto. Other than the Phyllostachys pubescens extract, the pharmaceutical composition of the present invention may further comprise a pharmaceutically acceptable carrier, vehicle, or diluent.

The pharmaceutical composition of the present invention may be produced in any one formulation selected from a capsule, a powder, a granule, a tablet, a suspension, an emulsion, a syrup, and an aerosol, but it is not limited thereto.

The pharmaceutical composition of the present invention can be administered either orally or parenterally. In the case of parenteral administration, it is preferable to choose an external application on the skin, or intraperitoneal, rectal, intravenous, muscular, subcutaneous, endometrium injection, or intracerebroventricular injection. Most preferably, it is used for external application on the skin.

The pharmaceutical composition of the present invention may be produced by using a diluent or a vehicle such as a filler, bulking agent, binding agent, moisturizing agent, disintegrating agent, or surfactant. Examples of solid preparation for oral administration include a tablet, a pill, a powder, a granule, and a capsule. The solid preparation is produced by mixing at least one compound with one or more vehicles such as starch, calcium carbonate, sucrose, lactose, or gelatin. Furthermore, other than simple vehicles, a lubricating agent such as magnesium stearate or talc is also used. As for the liquid preparation for oral administration, a suspension, a solution preparation for internal use, an emulsion, a syrup preparation, or the like can be mentioned. Other than water or liquid paraffin commonly used as a simple diluent, various kinds of a vehicle such as moisturizing agents, sweetening agents, aromatic agents, or preservatives may be included. Examples of a preparation for parenteral administration include a sterilized aqueous solution, a non-soluble preparation, a suspension preparation, an emulsion preparation, a freeze-dried preparation, and a suppository preparation. As a water-insoluble solvent or a suspending preparation, propylene glycol, polyethylene glycol, or vegetable oil such as olive oil, and injectable ester such as ethyl oleate can be used. As a base for a suppository preparation, witepsol, macrogol, tween 61, cacao fat, laurin fat, glycerol gelatin, or the like can be used.

The pharmaceutical composition of the present invention is administered in a pharmaceutically effective amount. As described herein, the expression “pharmaceutically effective amount” means an amount sufficient for treating a disorder at a reasonable benefit-risk ratio that can be applied for a medical treatment. The effective dose level may be determined based on a type or severeness of a disorder of a patient, the activity of a pharmaceutical, sensitivity to a pharmaceutical, administration period, administration route, excretion ratio, time period for therapy, elements including a pharmaceutical used in combination, and other elements that are well known in the medical field. The composition of the present invention can be administered as a separate therapeutic agent, or it can be used in combination with another therapeutic agent. It can be administered in order or simultaneously with a conventional therapeutic agent. It can be also administered as single-dose or multi-dose. It is important to administer an amount that allows obtainment of the maximum effect with a minimum dose while considering all of the aforementioned elements without having any side effect, and the dosage can be easily determined by a person skilled in the pertinent art.

The dosage of the composition of the present invention may vary depending on body weight, age, sex, health state, the diet of a patient, administration period, administration method, excretion rate, and severity of the disorder.

The present invention still further relates to a cosmetic composition for preventing or ameliorating androgen-dependent alopecia comprising Phyllostachys pubescens extract as an effective component.

The cosmetic composition of the present invention may be a hair cleansing composition, and it may be formulated into a hair shampoo, a hair rinse, a hair conditioner, a hair treatment, a hair tonic, a scalp treatment, a hair lotion, a hair cream, a nutritional hair toner, or a common ointment.

Hereinbelow, the present invention is explained in greater detail in view of the Examples. However, the following Examples are given only for specific explanation of the present invention and it would be evident to a person who has common knowledge in the pertinent art that the scope of the present invention is not limited by them.

### EXAMPLES

### Example 1. Culture of Human Benign Prostatic Hyperplasia Cell Line (BPH-1)

Human benign prostatic hyperplasia epithelial cells (BPH-1) were cultured in 5% CO₂ incubator at 37° C. using RPMI1640 containing 20% FBS and 1% antibiotics. According to subculture every 48 hours, cell buoyancy was maintained within a range of 70 to 90%. One day before carrying out the test, the cells were aliquoted and cultured for 24 hours for stabilization.

### Example 2. Preparation of Phyllostachys pubescens Extract

At room temperature, 1 kilogram of leaves of Phyllostachys pubescens Mazel (Phyllostachys edulis, Phyllostachys pubescens) was immersed in 15 liters of 80% (v/v) ethanol for 1 week and then extracted for 5 hours at 80° C. followed by filtration. After that, the extracted solution was concentrated under reduced pressure to give Phyllostachys pubescens extract, which was then used for Examples 4 to 6.

One kilogram of leaves of Phyllostachys pubescens Mazel (Phyllostachys edulis, Phyllostachys pubescens) was extracted with 10 liters of 80% ethanol for 3 hours at 80° C. followed by filtration. After that, the extracted solution was concentrated under reduced pressure to give Phyllostachys pubescens extract, which was then used for Example 7.

### Example 3. Establishment of the Luciferase Assay System

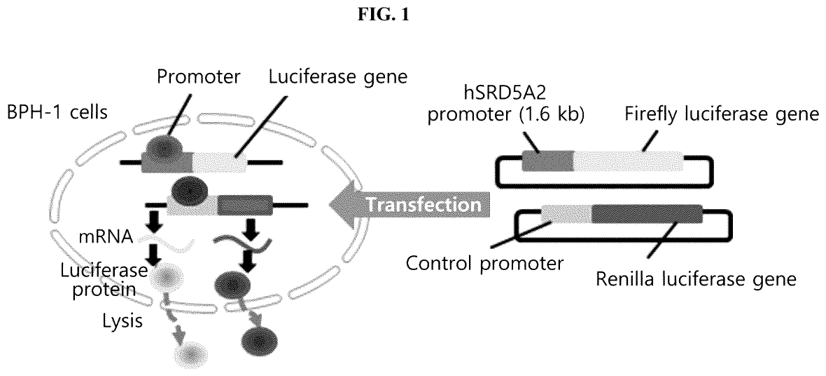

To determine a change in the expression amount of the SRD5A2 gene, the luciferase assay system was established (FIG. 1). Specifically, after cloning the promoter (about 1.6 kb) of human SRD5A2 gene in firefly luciferase reporter, it was subjected to transient transfection using BPH-1 cells. Then, the luciferase assay system for analyzing the luciferase reporter activity by measuring the luminescence intensity from firefly luciferase was established.

### Example 4. Screening Using Change in Transcription Activity in the Promoter Region of the SRD5A2 Gene

To select a candidate group capable of reducing the expression amount of the SRD5A2 gene from the library of oriental herb medicines, the luciferase assay established in above Example 3 was carried out.

The luciferase reporter containing human SRD5A2 promoter (about 1.6 kb) was subjected to transient transfection using BPH-1 cells, which were then treated with the library of various oriental herb medicines (Phyllostachys pubescens (Phyllostachys pubescens Mazel, Phyllostachys edulis) extract, Lophatheri herba (Lophatherum gracile Brongn.) extract, Bambusae caulis extract, and Sasa borealis extract) followed by overnight culture. After that, by measuring the firefly luciferase luminescence intensity, the candidate group capable of reducing the expression amount of the SRD5A2 gene was identified.

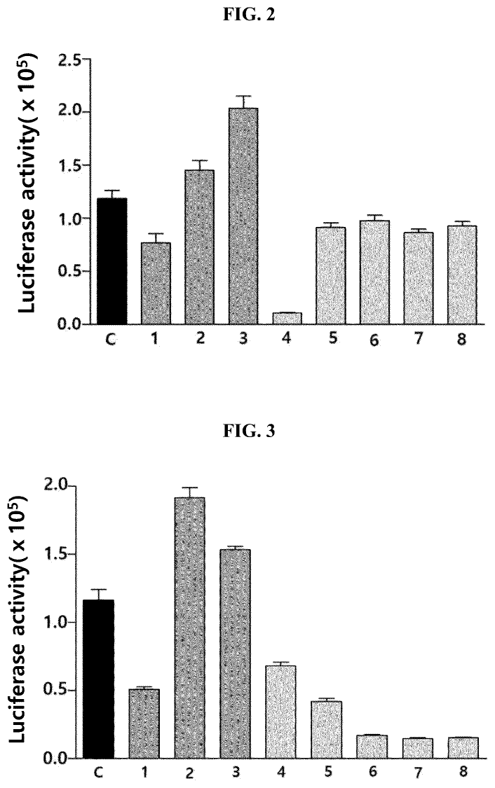

As the result is illustrated in FIG. 2, the group treated with the Phyllostachys pubescens extract of the present invention showed a significant decrease in the expression of SRD5A2. On the other hand, from the groups treated with an extract of other types of bamboo (i.e., Lophatheri herba, Bambusae caulis, Sasa borealis), the effect of reducing SRD5A2 expression was not observed.

As the result is illustrated in FIG. 3, it was also found that, when the treatment is carried out with the Phyllostachys pubescens extract of the present invention at various concentrations (i.e., 1 to 100 μg/ml), the SRD5A2 expression is reduced in concentration-dependent manner.

### Example 5. Determination of Change in Expression Amount of SRD5A2 Gene Caused by Treatment with Phyllostachys pubescens Extract

To analyze a change in the expression amount of the SRD5A2 gene that is caused by a treatment with the Phyllostachys pubescens extracts of the present invention, a treatment with Phyllostachys pubescens extract at a concentration of 3, 10, or 30 μg/ml was carried out followed by culture for 24 hours. Thereafter, total RNA was isolated from the cells and cDNA synthesis was carried out using reverse transcription polymerase chain reaction (RT-PCR). Based on real-time PCR using the TaqMan probe, a change in the expression amount of the SRD5A2 gene was then analyzed.

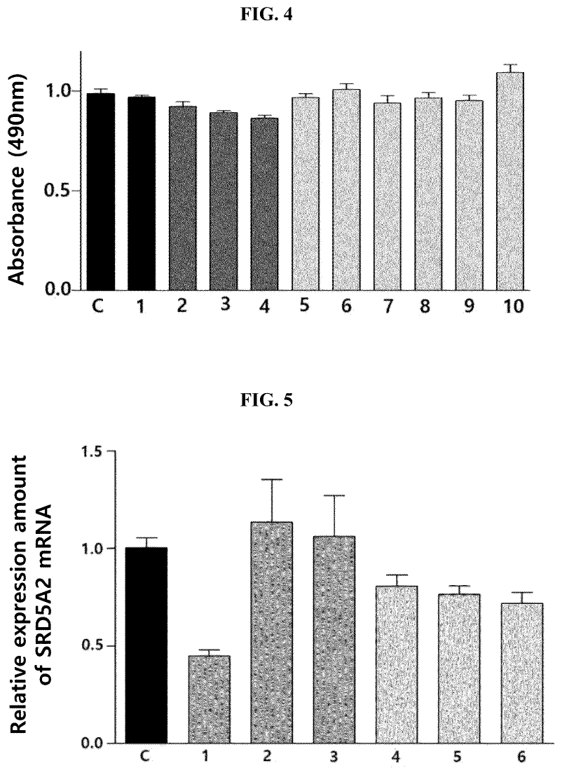

As the result is shown in FIG. 4, it was found that the expression of the SRD5A2 gene is reduced in the group which has been treated with 3 to 30 μg/ml Phyllostachys pubescens extract.

### Example 6. Determination of Cytotoxicity of Phyllostachys pubescens Extract

To determine the cytotoxicity of the Phyllostachys pubescens extract, cells were treated, in a concentration-dependent manner, with Phyllostachys pubescens extract, and then the cell viability was examined.

Specifically, BPH-1 cells were applied in a 48-well plate at a density of 2×10⁴ to 3×10⁴ cells/well. After culture for 24 hours, the cells were treated for 24 hours with 1 to 300 μg/ml Phyllostachys pubescens extract. Upon the completion of the reaction, the cells were added with MTS reagent, and further cultured for 4 hours. By using a microplate reader, absorbance was measured at a wavelength of 490 nm to measure cell viability.

As the result is shown in FIG. 5, it was found that the cell viability of the group treated with 1 to 300 μg/ml Phyllostachys pubescens extract is at a similar level as the control. Thus, it is recognized that the Phyllostachys pubescens extract has no cytotoxicity.

### Example 7. Determination of Change in Prostate Caused by Administration of Phyllostachys pubescens Extract

### [Animal Test]

A male rat (7-week-old, 280±10 g) was purchased from Daehan Bio Link Co., Ltd. The test animals were supplied with a sufficient amount of solid feed (not added with any antibiotics, Samyang Feed Co.) and water until the day of the experiment. Before the test, the animals were first acclimated for 1 week under an environment with a temperature of 22±2° C., humidity of 55±15%, and light/dark cycles of 12 hours. For having ethical and scientific considerations and efficient management, the animal experiment was carried out after obtaining the approval of the Animal Test Ethics Committee (Approval No.; DJUARB2020-017). The experiment animals were randomly divided into 5 groups (n=8) in total, including the control (CON); the group induced with benign prostatic hyperplasia by treatment with 5 mg/kg testosterone (BPH); and Phyllostachys pubescens extract treatment group treated with 50, 100 or 200 mg/kg Phyllostachys pubescens extract (PPE).

Rat after the acclimation period was anesthetized. After cutting the scrotum skin of the rat, the sperm tube, blood vessel, and nerve tissues were ligated by a thread, and the testis and epididymis were removed. After the removal, the scrotum skin was sutured and the animal was allowed to undergo a recovery period.

From Day 2 after the orchiectomy, all test animals except the control were subjected to subcutaneous injection of testosterone propionate (5 mg/kg), once a day for 4 weeks, to induce enlarged prostate. The Phyllostachys pubescens extract treatment group was orally administered with Phyllostachys pubescens extract at a concentration of 100, or 200 mg/kg, once a day for 4 weeks, together with subcutaneous injection of testosterone.

After that, the animal was sacrificed. Blood was taken from the animal and prostate tissues were separated and weighed. Based on H&E staining, a histological change and epithelial cells of the prostate tissues were examined.

(1) Effect of Administration of Phyllostachys pubescens Extract on Content of Testosterone, Dihydrotestosterone, PSA (Prostate Specific Antigen), and SRD5A2 in Blood Serum

For each test group, the content of testosterone, DHT, PSA, and SRD5A2 in blood serum was analyzed by ELISA using the ELISA kit of Mybiosource (USA).

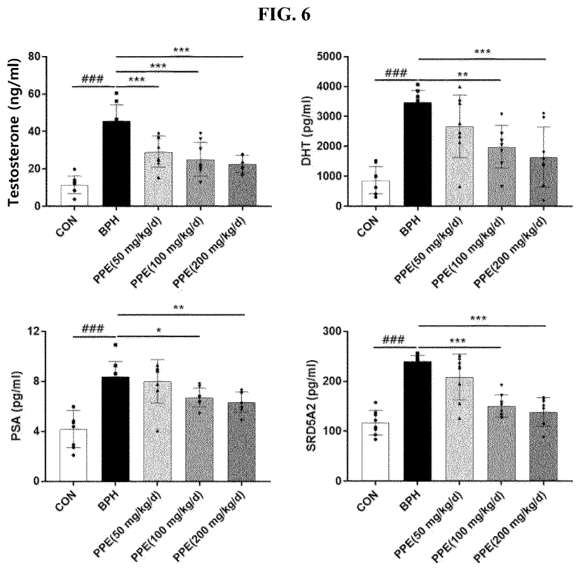

As a result, compared to the control (CON), a statistically significant increase in the content of testosterone, DHT, PSA, and SRD5A2 was shown from the BPH group. On the other hand, compared to the BPH group, a statistically significant decrease in the content of testosterone, DHT, PSA, and SRD5A2 in blood serum was shown from the Phyllostachys pubescens extract administration group of the present invention (PPE) (FIG. 6).

(2) Change in Prostate Weight Caused by Administration of Phyllostachys pubescens Extract

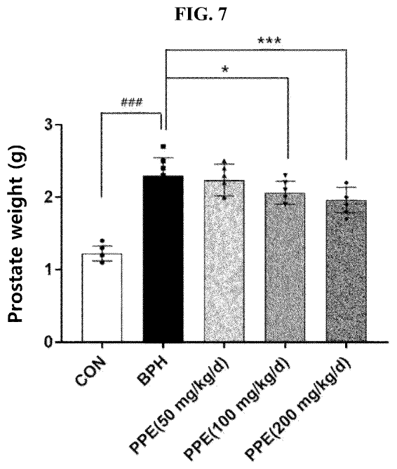

As a result of examining a change in prostate weight caused by the administration of Phyllostachys pubescens extract, it was found that, compared to the control, a statistically significant increase in prostate weight is obtained from the BPH group which has been induced to have an enlarged prostate. On the other hand, compared to the group which has been induced to have enlarged prostate (BPH), a statistically significant decrease in prostate weight was shown from the Phyllostachys pubescens extract administration group of the present invention (PPE) (FIG. 7).

(3) Morphological Change in Prostate Tissues Caused by Administration of Phyllostachys pubescens Extract

The prostate tissues were fixed in 4% paraformaldehyde and embedded in paraffin to give a specimen. After performing H&E staining of the specimen, a histological change in epithelial cells and stroma of the prostate was examined with an optical microscope.

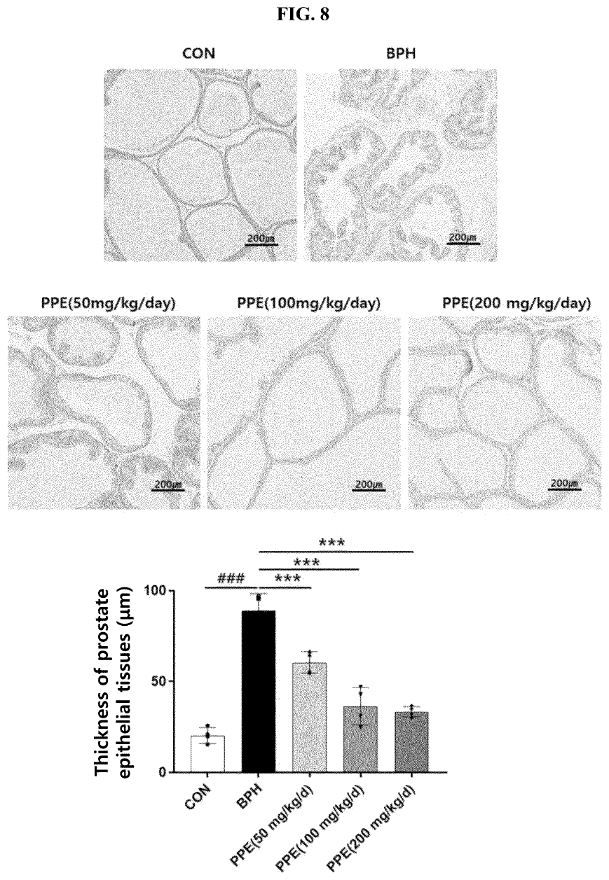

As the result is shown in FIG. 8, it was found that, in the prostate tissues of the control (CON), single-layer short columnar epithelial cells form secretory luminal cells and the lumen is filled with slight acidophilic materials. From the group which has been induced to have an enlarged prostate by testosterone (BPH), a hyperplastic condition showing higher epithelial thickness of the prostate was observed and fibrovascular proliferation was identified.

On the other hand, compared to the BPH group, from the PPE group which has been treated with the Phyllostachys pubescens extract of the present invention, reduced epithelial thickness of the prostate was observed and no stroma proliferation was shown. As such, it was found that the Phyllostachys pubescens extract of the present invention can reduce the proliferation of epithelial cells in prostate tissues and is effective for treating prostatic hyperplasia.

(4) Determination of Change in TNF-α Expression Amount in Prostate Tissues Caused by Administration of Phyllostachys pubescens Extract

The prostate tissues were collected and cut into a 20-μm thick specimen by using a cryostat microtome. After adhering the specimen to a slide, immune histology fluorescent (IHF) staining was carried out. Specifically, to fix the tissues, 4% paraformaldehyde (Sigma-Aldrich, USA) and 4% sucrose (Sigma-Aldrich, USA) were added and the fixing was carried out 45 minutes at room temperature. 10 mM Glycine (Sigma-Aldrich, USA) was further added to PBS-T (PBS solution added with 0.1% Triton X-100). After washing three times, each for 5 minutes, 0.5% NP-40 (Sigma-Aldrich, USA) was added to PBS and the reaction was allowed to occur for 30 minutes at room temperature. After washing for 5 minutes with PBS-T, a blocking reaction was carried out overnight at 4° C. by using a blocking buffer in which 5% goat serum (Biowest, USA), 5% horse serum (Biowest, USA), and 3% bovine serum albumin (Sigma-Aldrich, USA) are present as a mixture in PBS-T solution.

The primary antibody was diluted in the blocking buffer, and the reaction was allowed to occur for 4 hours at room temperature. Then, washing was carried out three times with PBS-T for 10 minutes each. After that, the secondary antibody was also diluted in blocking buffer, and the reaction was allowed to occur for 2 hours at room temperature under conditions in which light was blocked followed by one washing for 10 minutes with PBS-T.

For nuclear staining, the reaction was allowed to occur with Hoechst 33258 in PBS-T solution. After washing for 10 minutes with PBS-T, a cover glass (22×50 mm, Marienfeld-superior, Germany) was applied using GEL/MOUNT (Biomeda, USA) and dried in a dark room at room temperature. As for the primary antibody used for the staining, TNFα (Abcam, UK) was used. As for the secondary antibody, fluorescein goat anti-mouse IgG antibody (H+L) (Invitrogen, USA) and rhodamine goat anti-rabbit antibody (Invitrogen, Carlsbad, USA) were used. By using a fluorescence camera (fluorescence microscope, Nikon, Japan) and ACT-1 software, images were obtained from the stained slide.

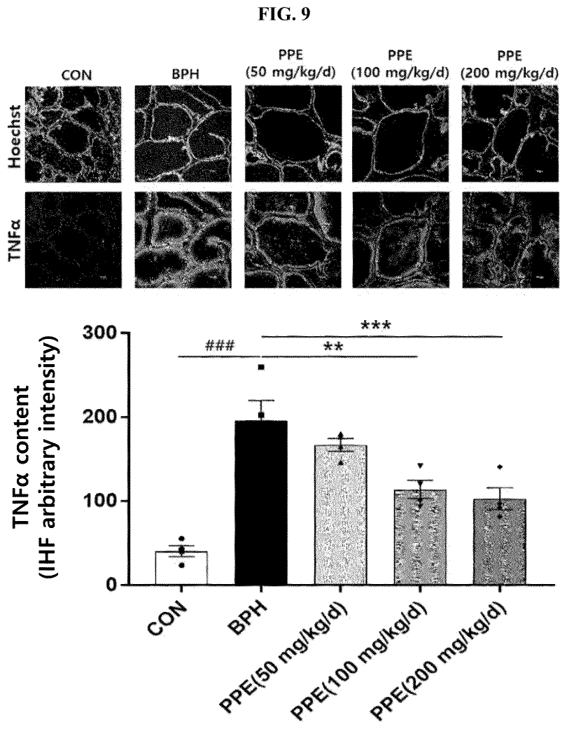

As the result is shown in FIG. 9, a significant increase in TNF-α expression in prostate tissues was shown from the testosterone administration group (BPH) compared to the normal group (Con). On the other hand, in the prostate tissues of the group administered with the Phyllostachys pubescens extract of the present invention (PPE), a statistically significant decrease in TNF-α expression was obtained compared to the testosterone administration group (BPH).

### [Statistical Processing]

All the data of the animal test were subjected to statistical analysis, and the data are expressed in the mean of the tested animals±standard deviation (SD). The statistical significance determination was made by statistical analysis based on Dunnett Post hoc test or ANOVA according to Uncorrected Fisher's LSD, in which the analysis has been made by using GraphPad Prism 7.05 software. When p-value is less than 0.05, it was considered that the data have a statistically significant difference.

1-8. (canceled)

9: A method for ameliorating or treating an androgen-dependent disorder, the method comprising administering a composition comprising Phyllostachys pubescens extract as an effective component to a subject in need thereof.

10: The method of claim 9, wherein the Phyllostachys pubescens extract is an extract of Phyllostachys pubescens by using an extraction solvent selected from the group consisting of water, a C₁-C₄ lower alcohol, and a mixture thereof.

11: The method of claim 9, wherein the Phyllostachys pubescens extract is an extract from a leaf of Phyllostachys pubescens Mazel.

12: The method of claim 9, wherein the androgen-dependent disorder is any one selected from the group consisting of alopecia, acne, seborrheic dermatitis, prostatitis, benign prostatic hyperplasia, dysuria caused by benign prostatic hyperplasia, and a combination thereof.

13: The method of claim 9, wherein the Phyllostachys pubescens extract is prepared in any formulation selected from the group consisting of a drink, a pill, a tablet, a capsule, and a powder.

14: The method of claim 9, wherein the composition is prepared in a functional health food.

15: The method of claim 9, wherein the composition is prepared in a pharmaceutical composition.

16: The method of claim 15, wherein the pharmaceutical composition further comprises at least one of a carrier, a vehicle, and a diluent.

17: The method of claim 9, wherein the composition is prepared in a cosmetic.

18: The method of claim 10, wherein the composition is prepared in a cosmetic.

19: The method of claim 17, wherein the androgen-dependent disorder is alopecia.

20: The method of claim 18, wherein the androgen-dependent disorder is alopecia.
